(19) United States
(12) Patent Application Publication
(10) Pub. No.: US 2026/0256356 A1
(43) Pub. Date: Sep. 3, 2026

(54) INTRAOCULAR PRESSURE MEASURING DEVICE AND METHOD

(71) Applicant: MINGCHE BIOTECHNOLOGY CO., LTD, Suzhou (CN)

(72) Inventors: Kemin Wang, Suzhou (CN); Dongni Ren, Suzhou (CN); Lijun Su, Suzhou (CN); Chuanyan Xu, Suzhou (CN)

(21) Appl. No.: 19/653,128

(22) Filed: Apr. 21, 2026

(30) Foreign Application Priority Data
Apr. 23, 2025 (CN) 202510512356.1

(51) Int. Cl. A61B 3/16; A61B 3/00; G06T 7/00; G06T 7/136

(52) U.S. Cl. CPC A61B 3/16; A61B 3/0008; A61B 3/0025; G06T 7/0012; G06T 7/136; A61B 2560/0223; G06T 2207/10004; G06T 2207/20081; G06T 2207/20084; G06T 2207/20112; G06T 2207/30041

(57) ABSTRACT

Disclosed is an intraocular pressure measuring device and method. The measuring method includes: identifying and segmenting interference fringe areas using the YOLOv11-seg model; conducting binary segmentation and extracting the interference fringe areas using a deep convolutional neural network; extracting skeletons of the binarizing interference fringes, marking orders of the fringes, and then calculating central deflections of a film of the Fabry-Pérot cavity under pressure; and acquiring intraocular pressures according to the central deflections. Square interference fringe areas are accurately identified, segmented, and extracted using the YOLOv11-seg model. Then, the skeletons of the fringes after binarization are extracted, the orders of the fringes are marked, and the central deflections of the film under pressure are calculated. Finally, according to the correspondence relation between the central deflection of the sensor and the pressure on the sensor calibrated, the pictures containing interference fringe images are converted into pressure values.

### CROSS-REFERENCE TO RELATED APPLICATIONS

The present application claims the benefit of Chinese Patent Application No. 202510512356.1 filed on Apr. 23, 2025, the contents of which are incorporated herein by reference in their entirety

### TECHNICAL FIELD

The present disclosure relates to the technical field of medical devices, and particularly to an intraocular pressure measuring device and method.

### BACKGROUND

Glaucoma, as one of the three major causes of blindness in human eyes, is extremely harmful. High intraocular pressure is considered an important risk factor for the onset of glaucoma. Therefore, intraocular pressure is an important indicator in clinical practice to determine the treatment goals of glaucoma and to evaluate the treatment effect and prognosis.

At present, the main means of measuring intraocular pressure is to measure the patient's immediate intraocular pressure with instruments. The measurement instruments mainly include applanation tonometers and air-puff tonometers. Applanation tonometers have a complicated measurement process, with disadvantages such as requiring surface anesthesia before measurement, dripping sodium fluorescein on the cornea during measurement, and the measured value being affected by central corneal thickness. Compared with applanation tonometers, air-puff tonometers feature simpler processes of intraocular pressure measurement and do not require topical anesthesia or sodium fluorescein. However, air-puff tonometers also have problems, including patient eye discomfort caused by the impact of the airflow, high cost, and limited portability.

In addition, there are many studies on micro-implantable intraocular pressure sensors based on different principles. These studies share the following characteristics: (1) the sensor is separated from the detection device, and the sensing method is non-contact; (2) the sensor area and volume are small, ranging from hundreds of microns to a few millimeters; and (3) the sensor contacts the eye structure and is attached to the eyeball or implanted inside the eyeball.

According to the sensing principle, these implantable intraocular pressure sensors are mainly classified into three types, namely electrical sensing, microfluidic sensing, and optical sensing. Chen et al. designed a contact lens-based intraocular pressure sensor that is sensitive to pressure based on capacitance. The frequency of the LC oscillator formed by capacitance and inductance also changes with the pressure change, and the reading device is a large network analyzer. Agaoglu et al. used microfluidic chips to detect intraocular pressure, and implanted an artificial lens integrated with a microfluidic chip into the eyeball using cataract surgery technology. As the intraocular pressure fluctuated, the position of the liquid-gas interface of the artificial lens shifted, and the intraocular pressure value could be acquired by monitoring the position of the interface. For electrical sensing, limited by the circuit structure and materials, it is difficult to reduce the sensor size to sub-millimeter level, and the reading devices are large and expensive. For microfluidic sensing, the stringent requirements for airtightness and the indirect sensing principle of photographic readings result in a bottleneck for miniaturization. For optical sensing, optical sensors are generally smaller than electrical sensors and microfluidic sensors, so the research on MEMS intraocular pressure sensors based on optical sensors has become the main direction of the research on implantable sensors.

The measurement solutions with MEMS intraocular pressure sensors based on optical sensing in the prior art are all based on the Fabry-Pérot interference structure, interference fringe patterns are captured, then interference areas are identified and extracted, and finally the interference fringe patterns are converted into pressures. In these solutions, the interference area identification and extraction methods in the prior art usually involve manual image cropping, making it difficult to apply these methods to real-time continuous demodulation processes, which limits the application of intraocular pressure measurement.

### SUMMARY

In order to solve the problem of the prior art that the interference area identification and extraction methods applied to MEMS intraocular pressure sensors can hardly be applied for real-time continuous demodulation processes, the present disclosure provides an intraocular pressure measuring method, in which the YOLOv11-seg model is introduced to precisely identify, segment, and extract interference fringe areas contained in pictures. In this way, the measuring method, as it avoids manual image cropping, realizes identification and extraction of interference areas, which solves the problem of the prior art that the interference area identification and extraction methods usually involve manual image cropping, making it difficult to apply these methods to real-time continuous demodulation processes.

The technical solution adopted by the present disclosure to solve the technical problem is as follows:

An intraocular pressure measuring method, including the following steps:
S1: sensing changes in intraocular pressure using a Fabry-Pérot cavity of an intraocular pressure sensor;
S2: emitting rays of light using a shooting module to the Fabry-Pérot cavity, and acquiring pictures of interference patterns using a shooting element;
S3: identifying and segmenting interference fringe areas contained in the pictures using the YOLOv11-seg model to obtain the interference fringe areas;
S4: conducting binarizing segmentation and extracting the interference fringe areas using a deep convolutional neural network, to obtain binarizing interference fringes;
S5: extracting skeletons of the binarizing interference fringes, marking orders of the fringes, and then calculating central deflections of a film of the Fabry-Pérot cavity under pressure; and
S6: acquiring intraocular pressures according to the central deflections.

In some embodiments, the step of identifying and segmenting interference fringe areas contained in the pictures using the YOLOv11-seg model includes:
S31: acquiring pictures of interference patterns under different environmental conditions;
S32: manually annotating the interference fringe areas in the pictures using the Labelme plug-in to obtain a txt file as a set of tags corresponding to images in a training set; and
S33: taking some images as the training set and some images as the verification set for model training, to obtain interference fringe areas.

In some embodiments, the deep convolutional neural network includes a left path, a right path, an encoding path, and a decoding path.

In some embodiments, a loss function used in model training for the deep convolutional neural network includes a focal loss (FL) function and a multi-scale structural similarity (MS-SSIM).

In some embodiments, the FL function is defined as:

$${L}_{FL} ( p , y ) = {L}_{FL} ( {p}_{t} ) = - 0.5 {( 1 - {p}_{t} )}^{r} log ( {p}_{t} )$$

$$where {p}_{t} = \{ \begin{matrix}p & y = 1 \\ 1 - p & otherwise\end{matrix} ;$$

y∈{1,0} represents the true value for a sample; p∈[0,1] is the output of the model, that is, the predicted probability that the true value of the sample is 1; and r is the adjustment factor of the FL function.

In some embodiments, the MS-SSIM is defined as:

$$MS\_SSIM ( x , y ) = {[ {l}_{M} ( x , y ) ]}^{{α}_{M}} {\prod }_{j = 1}^{M} {{[ {c}_{j} ( x , y ) ]}^{{β}_{j}} [ {s}_{j} ( x , y ) ]}^{{γ}_{j}}$$

where M is the number of scales; α_{M}, βⱼ, γⱼ are the weights of I_{M}(x, y), cⱼ(x, y), sⱼ(x, y) respectively; and I_{M}(x, y), cⱼ(x, y), sⱼ(x, y) are the brightness similarity, contrast similarity, and structural similarity between x and y on the scales M, i, j respectively.

In some embodiments, a deflection distribution function of the film of the Fabry-Pérot cavity under pressure is:

$$w = {w}_{0} {( 1 - \frac{{x}^{2}}{{a}^{2}} )}^{2} {( 1 - \frac{{y}^{2}}{{a}^{2}} )}^{2} [ 1 + {c}_{1} ( \frac{{x}^{2} + {y}^{2}}{{a}^{2}} ) + {{c}_{2} ( \frac{{x}^{2} + {y}^{2}}{{a}^{2}} )}^{2} ]$$

where (x, y) are the coordinates of a point on the film, for which the origin is the center of the film; w₀ is the central deflection of the film; α is the side length of the film; and c₁, c₂ are two empirical parameters.

In some embodiments, the step of acquiring intraocular pressures according to the central deflections comprises: using a calibrating device to calibrate the central deflections of the film of the Fabry-Pérot cavity, and acquiring the intraocular pressures according to the calculated central deflections.

Another object of the present disclosure is to provide an intraocular pressure measuring device that measures intraocular pressure using the intraocular pressure measuring method described above.

In some embodiments, the intraocular pressure measuring device includes an intraocular pressure sensor, a shooting module, and a shooting element;
the intraocular pressure sensor is provided with a Fabry-Pérot cavity;
the shooting module consists of a housing, an optical path assembly arranged in the housing, and a light source arranged outside the housing;
the housing is provided with a shooting hole;
rays of light emitted by the light source are transmitted to the Fabry-Pérot cavity after passing through the optical path assembly, and form interference patterns;
the shooting element includes a lens; and
the shooting hole is suitable for cooperating with the lens to acquire the interference patterns through the shooting element.

The present disclosure has the following beneficial effects:

The intraocular pressure measuring method according to the present disclosure employs a deep learning method to accurately identify, segment, and extract square interference fringe areas contained in pictures using the YOLOv11-seg model. Then, the skeletons of the fringes after binarization are extracted, the orders of the fringes are marked, and the central deflections of the film of the Fabry-Pérot cavity under pressure are calculated. Finally, according to the one-to-one correspondence relation between the central deflection of the sensor and the pressure on the sensor calibrated by the calibration device, the pictures containing interference fringe images are converted into pressure values.

### BRIEF DESCRIPTION OF DRAWINGS

The present disclosure will be further described below with reference to figures and embodiments.

FIG. 1 is a flowchart of an intraocular pressure measuring method according to the present disclosure;

FIG. 2 is a flowchart of the specific process of converting images to pressures according to the present disclosure;

FIG. 3 is a schematic diagram of the training results of the YOLOv11-seg model according to the present disclosure;

FIG. 4 is a schematic diagram of the structure of a deep convolutional neural network model according to the present disclosure;

FIG. 5 shows images of some data sets used in training according to the present disclosure;

FIG. 6 shows changes in fringe pattern under different pressures according to the present disclosure;

FIG. 7 is a schematic diagram of the structure of an intraocular pressure measuring device according to the present disclosure;

FIG. 8 is an exploded view of an intraocular pressure sensor according to the present disclosure;

FIG. 9 is an exploded view of a shooting module according to the present disclosure;

FIG. 10 is a schematic diagram of the structure of an optical path assembly according to the present disclosure; and

FIG. 11 is a schematic diagram of the effect of shooting angle on the integrity of optical interference patterns according to the present disclosure;

FIG. 12 is a schematic diagram of a pressure rise process according to the present disclosure;

FIG. 13 is a photo of a pressure device according to the present disclosure.

### NUMERICAL REFERENCES

1—intraocular pressure sensor; 11—sensor body; 12—bracket; 121—mounting end; 122—fixing end; 1221—wide section; 1222—gradient section; 1223—narrow section; 1224—anti-slip structure; 123—drainage groove; 1231—first groove structure; 1232—second groove structure; 2—shooting module; 21—housing; 211—shooting hole; 22—optical path assembly; 221—beam splitter cube; 222—plano-convex lens; 223—narrowband filter; 23—light source; 24—clamp; 241—C-shaped element; 2411—mounting groove; 2412—through hole; 242—threaded connector; 3—shooting element; 31—lens.

### DETAILED DESCRIPTION OF THE EMBODIMENTS

The present disclosure will now be described in further detail. The embodiments described below are exemplary and are intended to explain the present disclosure, but should not be understood as limiting the present disclosure. Based on the embodiments of the present disclosure, all other embodiments obtained by ordinary technicians in the art without creative work belong to the scope of protection of the present disclosure.

In order to make the above objectives, features, and advantages of the present disclosure more obvious and understandable, specific embodiments of the present disclosure will be described in detail with reference to the drawings below.

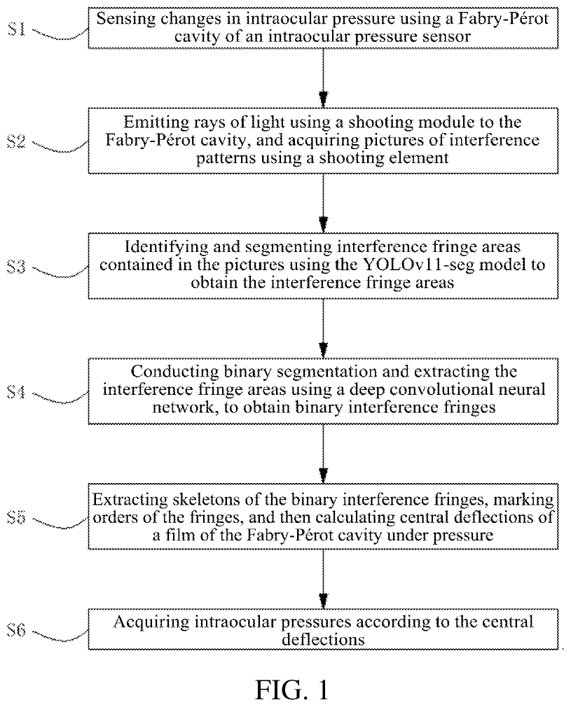

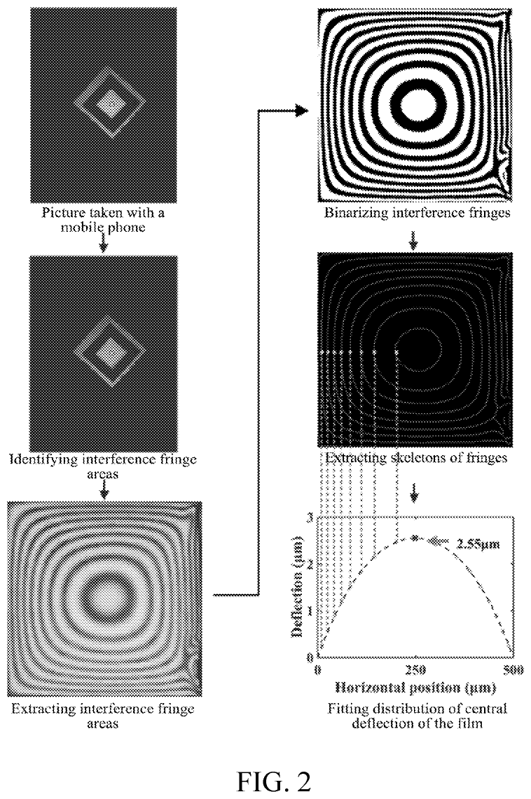

In order to solve the problem of the prior art that the interference area identification and extraction methods applied to MEMS intraocular pressure sensors can hardly be applied for real-time continuous demodulation processes, the present disclosure provides an intraocular pressure measuring method, which, taking the case that the shooting element is a mobile phone as an example, includes the following steps as shown in FIG. 1 and FIG. 2:
S1: sensing changes in intraocular pressure using a Fabry-Pérot cavity of an intraocular pressure sensor;
for the purpose of this step, a corresponding MEMS intraocular pressure sensor based on optical sensor can be implanted into the anterior chamber of the eyeball, making the outer surface of the Fabry-Pérot cavity contact the intraocular fluid to sense changes in intraocular pressure;
S2: emitting rays of light using a shooting module to the Fabry-Pérot cavity, and acquiring pictures of interference patterns using a shooting element;
the rays of light emitted from the shooting module come into the Fabry-Pérot cavity, where they are reflected by multiple surfaces, causing interference to form interference patterns;
specifically, the shooting module according to the present disclosure preferably includes an optical path assembly and a light source; the rays of light emitted by the light source are transmitted to the Fabry-Pérot cavity after passing through the optical path assembly, and form interference patterns; the shooting element includes a lens to obtain interference patterns using the shooting element;
preferably, the light source according to the present disclosure is a white light source; the shooting element may be any conventional digital camera, video camera, or smart phone including a CMOS image sensor; to further reduce the difficulty of shooting, preferably, a mobile phone is used as the shooting element according to the present disclosure; and a CMOS image sensor is provided in the mobile phone to realize the acquisition and imaging of interference patterns; and
during operation, rays of light emitted by the light source pass through the optical path assembly, and then normally come into the Fabry-Pérot cavity of the intraocular pressure sensor, which is an F-P resonator cavity, where the rays of light are reflected by multiple surfaces, causing interference to form interference patterns; and after passing through the optical path assembly, the obtained interference patterns are transmitted to the lens of the shooting element, and the shooting element takes pictures to capture the interference patterns in real time;
S3: identifying and segmenting interference fringe areas contained in the pictures using the YOLOv11-seg model to obtain the interference fringe areas;
the interference area identification and extraction methods in the prior art usually involve manual image cropping or employing the edge extraction technology to extract the area edge for further segmentation; manual image cropping makes it difficult to apply these methods to real-time continuous demodulation processes; the classical edge extraction technology, as different experiment conditions, different illumination intensities, different image resolutions, and other different environmental factors will result in different edge features of images, calls for manual adjustment of parameters to proper values, which is also unfavorable to real-time continuous measurement in many scenarios; in addition, interference fringes intrinsically have significant edge features, which further brings more instability of the conventional edge detection in this task;
based on these considerations, a deep learning method is employed in the present disclosure to accurately identify, segment, and extract square interference fringe areas contained in pictures using the YOLOv11-seg model, avoiding manual adjustment of parameters during extracting the interference fringes, so that the intraocular pressure measuring method according to the present disclosure can be applied to real-time continuous demodulation processes;
S4: conducting binarizing segmentation and extracting the interference fringe areas using a deep convolutional neural network, which avoids interference caused by noises in the images and uneven background brightness, to obtain binarizing interference fringes;
further, to extract the phase information carried by the interference fringes, a deep convolutional neural network is used in the present disclosure to further binarize the interference fringes in the interference areas;
S5: extracting skeletons of the binarizing interference fringes, marking orders of the fringes, and then calculating central deflections of a film of the Fabry-Pérot cavity under pressure; and
S6: acquiring intraocular pressures according to the central deflections.

The intraocular pressure measuring method according to the present disclosure employs a deep learning method to accurately identify, segment, and extract square interference fringe areas contained in pictures using the YOLOv11-seg model. Then, the skeletons of the fringes after binarization are extracted, the orders of the fringes are marked, and the central deflections of the film of the Fabry-Pérot cavity under pressure are calculated. Finally, according to the one-to-one correspondence relation between the central deflection of the sensor and the pressure on the sensor calibrated by the calibration device, the pictures containing interference fringe images are converted into pressure values.

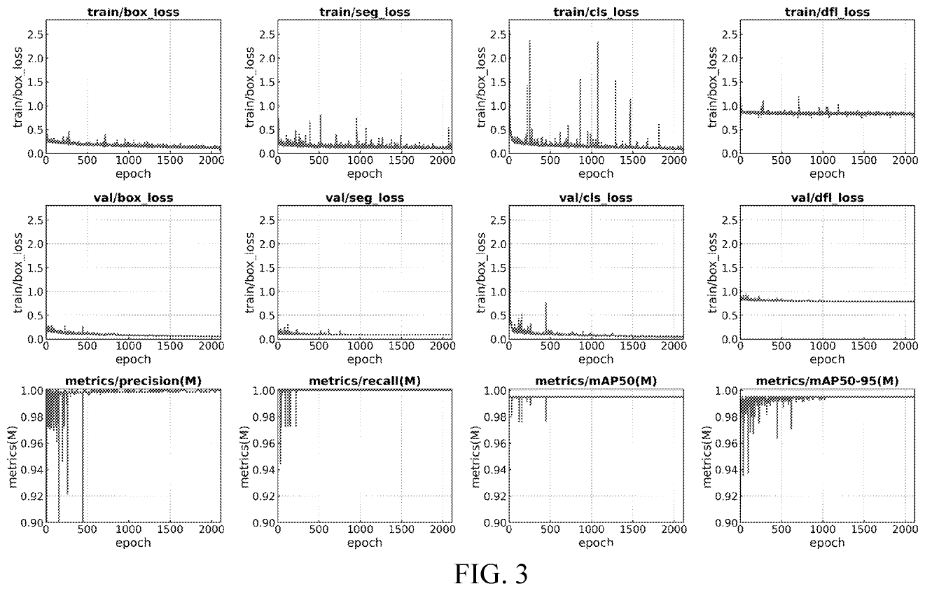

Specifically, the step of identifying and segmenting interference fringe areas contained in the pictures using the YOLOv11-seg model according to the present disclosure preferably includes:
S31: acquiring pictures of interference patterns under different environmental conditions;
taking the case that the shooting element is a mobile phone as an example, in this step, the mobile phone takes pictures containing interference fringe images of the sensors corresponding to different sensors, different pressures, different angles, different illumination intensities, different camera settings of the mobile phone, and other different environmental conditions;
S32: manually annotating the interference fringe areas in the pictures using the Labelme plug-in to obtain a txt file as a set of tags corresponding to images in a training set; and
the Label file exported from the Labelme plug-in is a json file by default, which is then converted to a txt file required by the YOLO model according to the corresponding rule, as a set of tags corresponding to images in a training set; these images and tags are then processed (for example, rotation, zooming, and translation) to further expand the data set;
S33: taking some images as the training set and some images as the verification set for model training, to obtain interference fringe areas;
during model training, preferably, 153 images are taken as the training set, and 54 images are taken as the verification set; the weight of the YOLOv11n-seg model pretrained is loaded, data augmentation is enabled during training, and the size of input image is specified to be 640×640×3; the performance indicators during training iteration are as shown in FIG. 3; and when the training is completed, the masked accuracy has reached over 99%.

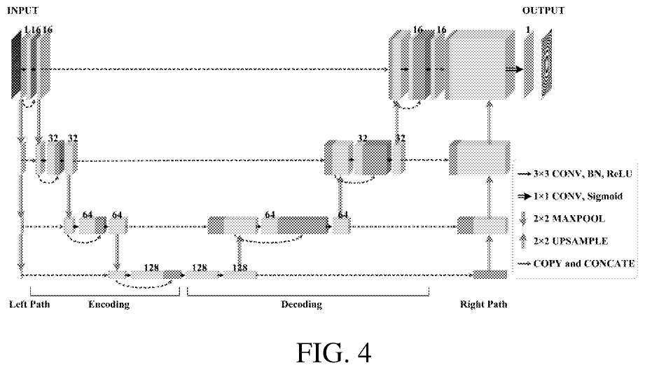

According to the present disclosure, the binarization of fringes is regarded as an image segmentation task, for which a model is used to segment bright fringes and dark fringes into two different classes. This model, as an improvement to the M-net applied to medical image segmentation, is a typical deep convolutional neural network, in which feature images of different sizes communicate with each other by means of skip connection on different scales, to achieve the effect of multi-scale feature fusion. The deep convolutional neural network contains a total of four paths, namely a left path, a right path, an encoding path, and a decoding path, among which the left and right paths are for deep supervision. The entire network is composed of convolution layers, a max-pooling layer, an up-sampling layer, a batch normalization layer, a ReLU layer, and a Sigmoid layer. Each path in the network includes feature images of four scales. The encoding path is designed with a typical CNN architecture. At each layer, two 3×3 Conv-BN-ReLU modules are first used to extract features, and then the 2×2 max-pooling layer with a step length of 2 reduces the size of each feature image to half of its original size. Therefore, the number of internal convolution kernels in every convolution layer is set as twice that in the previous convolution layer. The decoding path is designed with the completely same structure as the encoding path. In the decoding path, the structure and convolution layer parameters of each layer are set the same as those of the corresponding layer in the encoding path. The difference lies in that in the decoding path, a part of the 2×2 up-sampling layer is used for the inverse operation of the max-pooling layer to recover the feature images to their original sizes layer by layer, and finally, the output of the right path is cascaded with the output of the decoding path at the channel dimension, and is then sent to the 1×1 Conv-Sigmoid module to acquire the probability that each pixel is classified as a positive sample. At each layer, there are skip connections in the encoding path, in the decoding path, and between adjacent paths, for a better segmentation effect. The specific model structure is as shown in FIG. 4.

Further, a loss function used in model training for the deep convolutional neural network according to the present disclosure preferably includes a focal loss function and a multi-scale structural similarity; the loss function used in training this model is mainly composed of two parts, which are a focal loss (FL) function and a multi-scale structural similarity (MS-SSIM) respectively:

$${L}_{FL} ( p , y ) = {L}_{FL} ( {p}_{t} ) = - 0.5 {( 1 - {p}_{t} )}^{r} log ( {p}_{t} ) ;$$

Specifically, in the experiment, pixels at fringe edges tended to be segmented incorrectly, resulting in unsmoothed fringe edges. The FL function, as an improved cross-entropy loss function, is used to deal with the severe class imbalance between the foreground and the background during the training of the dense detector for target detection tasks. Preferably, the FL function according to the present disclosure is defined as:

$${L}_{FL} ( p , y ) = {L}_{FL} ( {p}_{t} ) = - 0.5 {( 1 - {p}_{t} )}^{r} log ( {p}_{t} )$$

$$where {p}_{t} = \{ \begin{matrix}p & y = 1 \\ 1 - p & otherwise\end{matrix} ;$$

y∈{1,0} represents the true value for a sample; p∈[0,1] is the output of the model, that is, the predicted probability that the true value of the sample is 1; and r is the adjustment factor of the FL function, and its value is set to 2 in the present disclosure.

Interference fringes have significant structural features. The human visual system can extract structural information from images. Therefore, structural similarity (SSIM) is a good measure for estimating the quality of images perceived by human beings. The SSIM between an image x and an image y is calculated with the equation below:

$$SSIM ( x , y ) = {{{[ l ( x , y ) ]}^{α} [ c ( x , y ) ]}^{β} [ s ( x , y ) ]}^{γ}$$

where α, β, and γ are the corresponding weight factors; and l(x, y), c(x, y), s(x, y) are the brightness similarity, contrast similarity, and structural similarity between the two images. However, the SSIM is calculated on a fixed scale, so it is only suitable for images on a certain scale. The MS-SSIM is an improvement to the SSIM. When the angle of view changes, the MS-SSIM is more flexible than the one-scale SSIM. The MS-SSIM according to the present disclosure is defined as:

$$MS\_SSIM ( x , y ) = {[ {l}_{M} ( x , y ) ]}^{{α}_{M}} {\prod }_{j = 1}^{M} {{[ {c}_{j} ( x , y ) ]}^{{β}_{j}} [ {s}_{j} ( x , y ) ]}^{{γ}_{j}}$$

where M is the number of scales; α_{M}, βⱼ, γⱼ are the weights of I_{M}(x, y), cⱼ(x, y), sⱼ(x, y) respectively; and I_{M}(x, y), cⱼ(x, y), sⱼ(x, y) are the brightness similarity, contrast similarity, and structural similarity between x and y on the scales M, i, j respectively.

The value range of the MS-SSIM is [0, 1], and the value is 1 when and only when the two images are exactly the same. Therefore, the MS-SSIM loss function is defined as:

$${L}_{MS - SSIM} ( p , y ) = 1 - MS\_SSIM ( x , y )$$

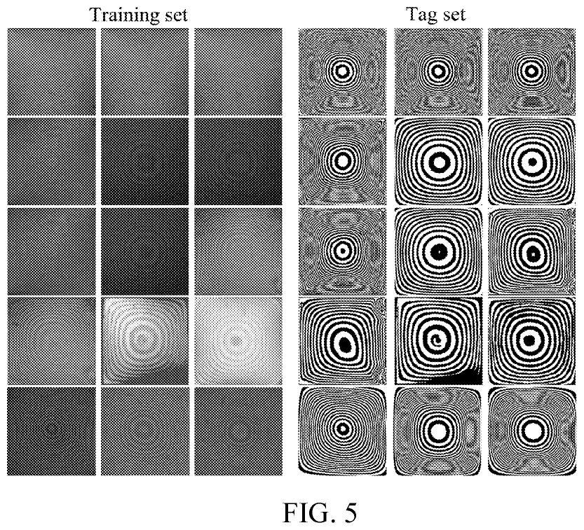

Further, the method for establishing the data set for the deep convolutional network according to the present disclosure is as follows: 26 interference fringe images captured during the experiment were taken as the training set, the fuzzy clustering mean (FCM) algorithm was used for binarization of these images, and then Adobe Photoshop was used to further process these binary images. As the number of images in the data set was too small, the images in the training set and the tag set were randomly processed, such as cropping, rotating, mirroring, perspective transformation, adding noise, changing brightness, and changing contrast, to obtain up to 2600 samples. In addition, MATLAB 2024a was used to generate 500 simulated interference images and corresponding binary images, and a total of 3100 496×496 images were taken as the data set for training. The device for training was equipped with 32 GB RAM, 12th Gen Intel (R) Core (TM) i7-12650H CPU, and NVIDIA Geforce RTX 4060 Laptop GPU. During training, the stochastic gradient descent (SGD) algorithm was used to optimize the network parameters. The size of processing batch was set as 4; the initial learning rate was set as 0.01; the Nesterov momentum was set as 0.75; the iterative attenuation rate of the learning rate was set as 0.00005, and it was designed to cease the training if the loss of the verification set does not drop after 5 epochs; and the maximum number of network training rounds was set as 50. Finally, 35 training rounds were completed, which took about 9 hours. Some images in the training set used in the training are as shown in FIG. 5.

Further, the skeletons of the binarizing interference fringe images were extracted, their orders were marked, and the corresponding unwrapped phases were calculated. The deflection distribution function of the film of the Fabry-Pérot cavity under pressure is:

$$w = {w}_{0} {( 1 - \frac{{x}^{2}}{{a}^{2}} )}^{2} {( 1 - \frac{{y}^{2}}{{a}^{2}} )}^{2} [ 1 + {c}_{1} ( \frac{{x}^{2} + {y}^{2}}{{a}^{2}} ) + {{c}_{2} ( \frac{{x}^{2} + {y}^{2}}{{a}^{2}} )}^{2} ]$$

where (x, y) are the coordinates of a point on the film, for which the origin is the center of the film; w₀ is the central deflection of the film; α is the side length of the film; and c₁, c₂ are two empirical parameters. In the demodulation calculation, only the central line of the film was used for fitting, so γ was 0, and the deflection shape function of the square film became the deflection distribution function of the central line of the square film:

$$w = {w}_{0} {( 1 - \frac{{x}^{2}}{{a}^{2}} )}^{2} [ 1 + {c}_{1} ( \frac{{x}^{2}}{{a}^{2}} ) + {{c}_{2} ( \frac{{x}^{2}}{{a}^{2}} )}^{2} ]$$

According to the present disclosure, the pixel coordinates of the central skeletons of the binarizing interference fringes and the values of the corresponding unwrapped phases were extracted to fit the deflection distribution of the central lines of the film, and further to obtain the deflections at the centers of the interference fringe images, namely the deflections at the center of the square film.

Further, according to the present disclosure, the central deflection of the sensor film demodulated from the pictures was associated to the pressure of the narrow cavity where the sensor was, that is, the intraocular pressure was acquired according to the central deflection, which includes: using a calibration device to calibrate the central deflections of the film of the Fabry-Pérot cavity under different pressures, and acquiring the intraocular pressure according to the calculated central deflection.

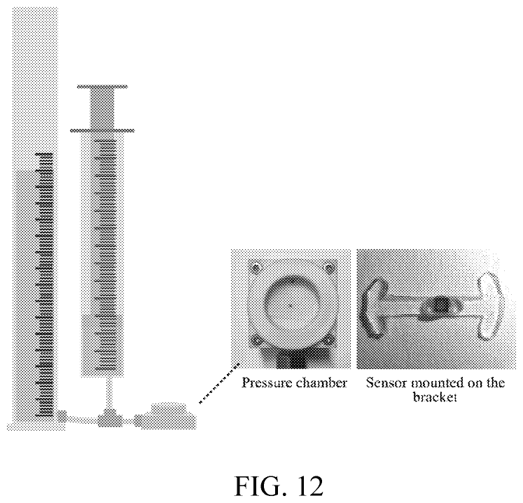

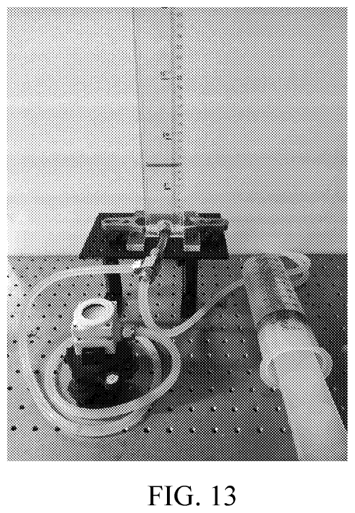

Specifically, as shown in FIG. 12, the cubic intraocular pressure sensor was fixed by an I-shaped silicone bracket, and the intraocular pressure sensor was mounted on the I-shaped bracket using ultraviolet light curing glue, and then the I-shaped bracket was further adhered to the pressure chamber. As shown in FIG. 13, in the experiment, a syringe was used as the pressure device. At the beginning of the experiment, the pressure chamber needs to be fixed at the same horizontal plane as the bottom of the measuring cylinder, to ensure that the pressure value in the pressure chamber is the liquid level displayed in the measuring cylinder. In the experiment, the liquid level of pure water in the measuring cylinder was controlled by pushing and pulling the syringe to control the hydraulic pressure on the intraocular pressure sensor in the pressure chamber.

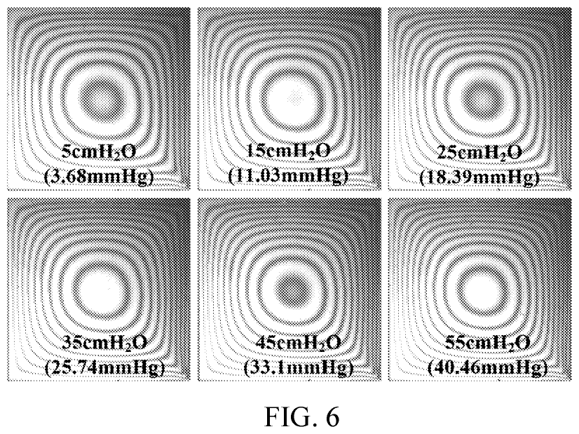

In the experiment, three rounds of reciprocating pressurization were carried out, and the pressure was held at 5 cmH₂O, 15 cmH₂O, 25 cmH₂O, 35 cmH₂O, 45 cmH₂O, and 55 cmH₂O (namely 3.68 mmHg, 11.03 mmHg, 18.39 mmHg, 25.74 mmHg, 33.1 mmHg, and 40.46 mmHg) for 3 min respectively, to ensure that the intraocular pressure sensor was in a stable hydraulic environment. The fringe images recorded during the experiment are shown in FIG. 6, which shows that the fringe density increases along with the rise of the pressure.

Then, the images recorded in the experiment were demodulated to plot the curves of the central deflection under different pressures, and a linear regression analysis was performed on the six curves. The determination coefficients R² of the curves of the three rounds of reciprocating pressurization are higher than 0.99, and the six curves show high repeatability. The final pressure sensitivity obtained by fitting the data of three rounds of reciprocating pressurization is 20.92 nm/mmHg.

Another object of the present disclosure is to provide an intraocular pressure measuring device that measures intraocular pressure using the intraocular pressure measuring method described above.

During the intraocular pressure measuring process, the intraocular pressure measuring device according to the present disclosure employs a deep learning method to accurately identify, segment, and extract square interference fringe areas contained in pictures using the YOLOv11-seg model. Then, the skeletons of the fringes after binarization are extracted, the orders of the fringes are marked, and the central deflections of the film of the Fabry-Pérot cavity under pressure are calculated. Finally, according to the one-to-one correspondence relation between the central deflection of the sensor and the pressure on the sensor calibrated by the calibration device, the pictures containing interference fringe images are converted into pressure values.

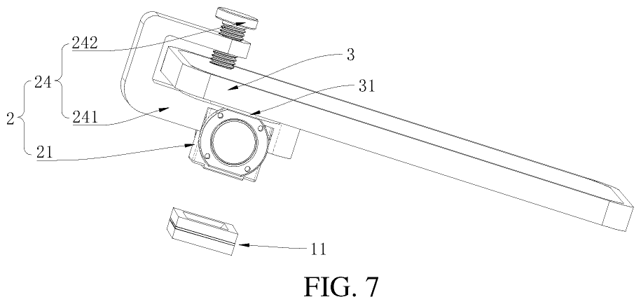

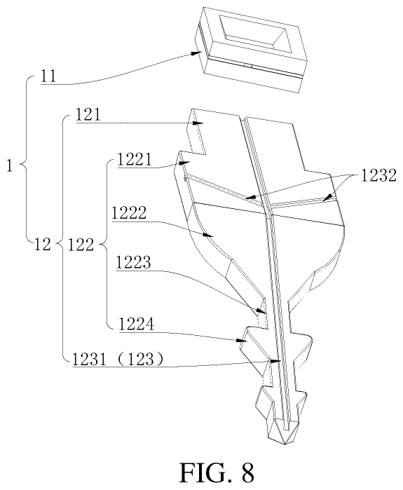

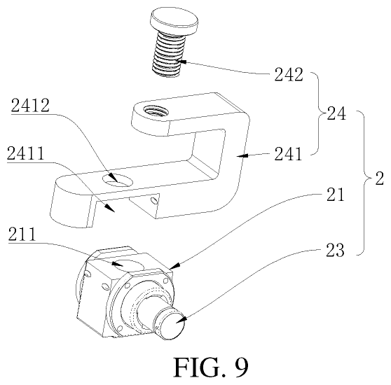

Specifically, as shown in FIG. 7, the intraocular pressure measuring device according to the present disclosure includes an intraocular pressure sensor 1, a shooting module 2, and a shooting element 3. It should be noted that, in FIG. 7, a zoom-in of the size of the intraocular pressure sensor has been made on purpose to clearly show the structure of the intraocular pressure measuring device. The intraocular pressure sensor 1 is a MEMS sensor, as shown in FIG. 8, and a Fabry-Pérot cavity is provided on the intraocular pressure sensor 1. As shown in FIG. 9, the shooting module 2 includes a housing 21, which is preferably made of polylactic acid (PLA), and prepared with an extrusion 3D printing process. The optical path assembly 22 is arranged in the housing 21, and the light source 23 is arranged outside the housing 21. The housing 21 is provided with a shooting hole 211. Rays of light emitted by the light source 23 are transmitted to the Fabry-Pérot cavity after passing through the optical path assembly 22, and form interference patterns. The shooting element 3 includes a lens 31. The shooting hole 211 is suitable for cooperating with the lens 31 to acquire the interference patterns through the shooting element 3.

Preferably, the light source 23 according to the present disclosure is a white light source.

During operation, rays of light emitted by the light source 23 pass through the optical path assembly 22, and then normally come into the Fabry-Pérot cavity of the intraocular pressure sensor 1, which is an F-P resonator cavity, where the rays of light are reflected by multiple surfaces, causing interference to form interference patterns. After passing through the optical path assembly 22, the obtained interference patterns are transmitted to the lens 31 of the shooting element 3, and the shooting element 3 takes pictures to capture the interference patterns in real time. Then, real-time intraocular pressures can be acquired according to the interference patterns captured in real time.

Specifically, the method for acquiring intraocular pressures according to interference patterns can be the method described above.

In actual use, the intraocular pressure sensor 1 is implanted in the anterior chamber of the eyeball, making the outer surface of the Fabry-Pérot cavity contact the intraocular fluid to sense changes in intraocular pressure. When the intraocular pressure increases, the Fabry-Pérot cavity deforms, causing the optical path of the reflected light to change, so that the generated interference pattern changes, and the interference fringes bend. By accurately identifying, segmenting, and extracting the interference fringe areas of the interference patterns, the skeletons of the fringes after binarization are extracted, the orders of the fringes are marked, and the central deflections of the film of the Fabry-Pérot cavity under pressure are calculated. Then, according to the one-to-one correspondence relation between the central deflection of the film of the Fabry-Pérot cavity and the pressure on the film of the Fabry-Pérot cavity calibrated by the calibration device, the pictures containing interference fringe images are converted into pressure values, and finally the intraocular pressure can be acquired according to the changes in the interference pattern.

With the shooting module 2 adapted to shooting elements 3 in the prior art, the intraocular pressure measuring device according to the present disclosure can detect the intraocular pressure with a shooting element 3 in the prior art. Furthermore, the positions of the shooting module 2 and the shooting element 3 can be adjusted according to the position of the Fabry-Pérot cavity in the intraocular pressure sensor 1, so the incident light beam fulfills the normal incidence requirement, significantly reducing the difficulty of angle adjustment during intraocular pressure detection, which makes the device more portable, efficient, and easy to use.

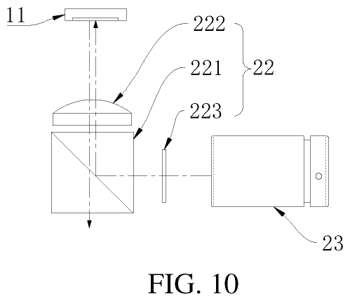

To achieve the measurement of intraocular pressure, as shown in FIG. 10, the optical path assembly 22 according to the present disclosure preferably includes a beam splitter cube 221 and a plano-convex lens 222 sequentially arranged in the shooting hole 211. The working wavelength range of the beam splitter cube 221 is from 450 nm to 650 nm. When the light comes in at an incident angle of 45°, the incident light can be divided into two beams of light at a ratio of about 50% transmission (T) and 50% reflection (R), with a tolerance of ±5% (T/R=50%: 50%±5%), so that the rays of light emitted by the light source 23 are reflected by the beam splitter cube 221 to adjust the optical path direction, and then converged by the plano-convex lens 222 to the target plane for generating optical interference patterns, where the Fabry-Pérot cavity is located. The reflected light of the interference pattern is then transmitted to the lens 31 of the shooting element 3 after passing through the plano-convex lens 222 and the beam splitter cube 221, for collecting and imaging the interference pattern. Preferably, the plano-convex lens 222 is designed with a wavelength of 350 nm to 700 nm and a focal length of 20 mm.

Further, the optical path assembly 22 according to the present disclosure preferably includes a narrowband filter 223 arranged between the light source 23 and the beam splitter cube 221, and the narrowband filter 223 is a 633 nm narrowband filter, so that the light emitted by the light source 23 is filtered by the narrowband filter 223 to obtain monochromatic light with a central wavelength of 633 nm and a bandwidth of ±10 nm.

The shooting element 3 according to the present disclosure can be any conventional digital camera, video camera, or smart phone including a CMOS image sensor. To further reduce the difficulty of shooting, preferably, a mobile phone is used as the shooting element 3 according to the present disclosure, with a CMOS image sensor provided in the mobile phone to realize the acquisition and imaging of interference patterns.

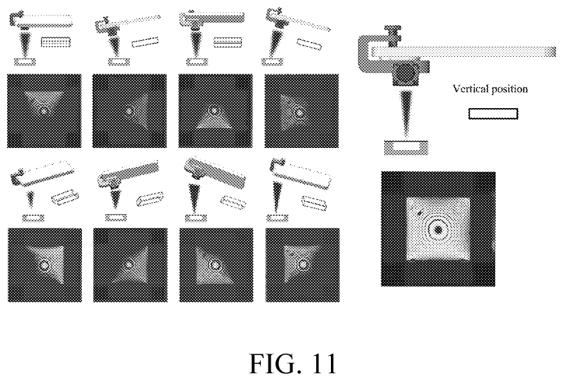

When an optical sensing-based pressure sensor in the prior art is used to measure the intraocular pressure, the incident light needs to fulfill the equal inclination interference, for which the main requirement is that the incident angle and the reflection angle (or the refraction angle) of the two interfering beams are equal when they are reflected or refracted. Specifically, the incident light beam needs to meet the requirement of normal incidence to ensure that the interference fringes are fully formed. When a micro pressure sensor is implanted in a pressure detection environment, it is impossible to guarantee that it is arranged horizontally due to its small size. When a desktop microscope is used, the microscope can generally only keep a vertical downward arrangement. When a complete optical interference pattern is expected, the only method is to adjust the spatial angle position of the object being measured by feel. This is extremely difficult, especially when the spatial angle position of the object being measured cannot be adjusted, for example, an intraocular pressure sensor implanted in the eye. Considering these issues, the present disclosure proposes an external shooting module 2 that can be adapted to any smart phone. Compared with adjusting the uncertain spatial angle position of the object to be measured, it is obviously much easier to adjust the angle of a mobile phone. Furthermore, the angle at which the mobile phone should be tilted can be determined according to the image captured in real time by its camera. The optical interference pattern captured in real time by the mobile phone camera is a square area. When the incident light is not normal to the interference plane, the square area is incomplete, appearing partially bright and partially dark. As shown in FIG. 11, the square interference area can be imagined as a sealed “square box” filled with water, and the bright part as a “bubble” in the sealed space. When the “bubble” is in a part of the square area, the “square box” will be tilted in the corresponding direction until the “bubble” is at the center of the square area. The mobile phone is exactly the “square box”. When the “bubble” is at the center of the square area, the incident light is in the normal direction to the interference plane. Then, the complete optical interference pattern is captured.

In order to facilitate connection with a mobile phone, preferably, the shooting module 2 according to the present disclosure further includes a clamp 24. One end of the clamp 24 is connected to the shooting element 3, namely the mobile phone, and the other end is connected to the housing 21.

Preferably, the clamp 24 according to the present disclosure is made of polylactic acid (PLA) and prepared with an extrusion 3D printing process. Further preferably, the clamp 24 is of a structure similar to a C-shaped clamp, including a C-shaped element 241 and a threaded connector 242. The C-shaped element 241 is connected to the housing 21. The C-shaped element 241 is connected to the shooting element 3 by a threaded connector 242. During use, the mobile phone is placed in the C-shaped element 241 and a connection is established by tightening the threaded connector 242. Preferably, the opening range of the C-shaped element 241 according to the present disclosure is 8 mm to 20 mm, which can be adapted to the thickness of most smart phones on the market.

Further, the housing 21 and the clamp 24 according to the present disclosure are preferably connected in a buckle manner. Specifically, a mounting groove 2411 adapted to the housing 21 is preferably provided on the C-shaped element 241 of the clamp 24, a concave point is provided in the mounting groove 2411, and a convex point adapted to the concave point is provided on the outer side of the housing 21. In this way, the two parts can be easily assembled or disassembled by engagement or disengagement of the convex point and the concave point.

In addition, a through hole 2412 adapted to the shooting hole 211 is provided in the mounting groove 2411, so that the C-shaped element 241 will not affect light transmission.

The present disclosure provides an external shooting module 2 compatible with any smart phone. The housing 21 of the external shooting module 2 is made of environmentally friendly polylactic acid (PLA), and a precisely controlled extrusion 3D printing process is employed to ensure the consistency of structural strength and quality. The shooting module 2 integrates an optimized customized optical path design and high-performance optical elements, combined with real-time image capture, to achieve stable shooting of high-quality images. Compared with the first generation of desktop microscope image capture methods, this module not only ensures image clarity and optical image quality, but also significantly reduces the complexity of user operation and minimizes the impact of jitter during shooting, making the device more portable and more efficient to operate, while improving its user experience and applicability.

The intraocular pressure sensor 1 according to the present disclosure can be any intraocular pressure sensor in the prior art that is provided with a Fabry-Pérot cavity. Since the intraocular pressure monitoring device according to the present disclosure detects the intraocular pressure based on optical sensing, as mentioned above, during detection, the incident light needs to meet the requirement of equal-inclination interference, which requires that the incident light beam meet the requirement of normal incidence. Therefore, in order to ensure the clarity of the detection image, the intraocular pressure sensor 1 is required to be at a fixed position and should not move during detection. In order to avoid the intraocular pressure sensor 1 from moving during detection, preferably, the intraocular pressure sensor 1 according to the present disclosure includes a sensor body 11 and a bracket 12 connected to the sensor body 11, so that the sensor body 11 can be fixed by the bracket 12, to reduce the difficulty of detection and improve the clarity of the detection image.

Brackets in the prior art used to fix intraocular implants are mostly cylindrical structures. For the intraocular pressure measuring device provided by the present disclosure, if the position of the sensor body 11 moves slightly during detection, the incident light beam will not normally come in, requiring readjustment of the shooting angle of the shooting element 3. Therefore, in order to ensure the stability of the position of the sensor body 11 during detection, preferably, the bracket 12 according to the present disclosure is of a plate-like structure to increase the contact area between the bracket 12 and the inside of the eye and avoid the movement of the sensor body 11.

Specifically, the bracket 12 according to the present disclosure preferably includes a mounting end 121 and a fixing end 122 connected to the mounting end 121. The mounting end 121 is used to connect to the sensor body 11, and the sensor body 11 and the mounting end 121 according to the present disclosure can be connected by methods such as high-temperature bonding and compatible material bonding. The size of the mounting end 121 is determined according to the size of the sensor body 11.

Since an eyeball has a certain curvature, in order to improve the fit between the bracket 12 and the eyeball, thereby enhancing the stability of the sensor body 11 and the patient's comfort, preferably, the fixing end 122 according to the present disclosure is of an arc-shaped structure, and the curvature of the arc-shaped structure is determined according to the curvature of the eyeball.

In order to improve comfort while ensuring the stability of the sensor body 11, preferably, the fixing end 122 according to the present disclosure includes a wide section 1221, a gradient section 1222, and a narrow section 1223 connected sequentially, and the widths of the wide section 1221, the gradient section 1222, and the narrow section 1223 decrease in sequence.

It should be noted that for the bracket 12 according to the present disclosure, the direction in which the wide section 1221, the gradient section 1222, and the narrow section 1223 are distributed is the length direction, and the direction perpendicular to the length direction on the plane of the plate-like structure of the bracket 12 is the width direction.

Specifically, the wide section 1221 according to the present disclosure is preferably of a plate-like structure with a rectangular cross section to ensure the contact area between the bracket 12 and the eyeball. Preferably, the width of the gradient section 1222 decreases gradually, so that the width of the end connected to the wide section 1221 is the same as the width of the wide section 1221, and the width of the end connected to the narrow section 1223 is the same as the width of the narrow section 1223.

In order to take into account both comfort and the position stability of the sensor body 11, preferably, the length ratio of the wide section 1221, the gradient section 1222, and the narrow section 1223 according to the present disclosure is (1.4 to 1.7):(1.1 to 1.4):(0.7 to 1).

In order to further improve the stability of the sensor body 11 after implantation, preferably, an anti-slip structure 1224 is provided on the outer side of the narrow section 1223 according to the present disclosure, and specifically, the anti-slip structure 1224 is preferably of a convex structure extending outward along the narrow section 1223.

In order to take into account the stability and comfort of the position of the intraocular pressure sensor body 11, further preferably, at least two groups of protrusion structures are provided according to the present disclosure, each group including two protrusions of the same size symmetrically arranged on the two sides of the narrow section 1223, and protrusion sizes gradually decrease in the direction away from the gradient section 1222.

Further preferably, the fixing end 122 according to the present disclosure is provided with a drainage groove 123, so that the intraocular pressure measuring device according to the present disclosure has both the intraocular pressure measurement function and the drainage function to a certain extent.

Intraocular pressure sensors in the prior art usually only have the function of intraocular pressure detection, but not the drainage function. When the intraocular pressure is high, a corresponding drainage device is required to achieve the treatment effect. Considering this issue, preferably, the bracket 12 according to the present disclosure is provided with a drainage groove 123, so that the aqueous humor can be diffused to the tissue around the eye through the drainage groove 123. In this way, the intraocular pressure sensor can have both intraocular pressure detection and drainage functions, so that intraocular pressure detection and drainage can be achieved through one implantation without increasing the number of implantations, surgically induced damage, or the patient's pain.

The drainage groove 123 includes a first groove structure 1231, which is a groove structure distributed along the longitudinal direction of the bracket 12 and sequentially penetrating the mounting end 121 and the fixing end 122. In order to further improve the drainage effect, the drainage groove 123 further includes a second groove structure 1232 obliquely distributed on the wide section 1221, and the second groove structure 1232 is connected to the first groove structure 1231.

The intraocular pressure sensor according to the present disclosure can be implanted into the eye by injection, significantly reducing the surgical trauma. With the intraocular pressure sensor according to the breakthrough design of the present disclosure that can be implanted into the eye by minimally invasive injection, the system can realize all-weather continuous monitoring of the intraocular pressure without electronic components or electromagnetic energy supply, and the intraocular pressure measurement accuracy reaches ±1 mmHg.

The intraocular pressure sensor according to the present disclosure can establish a functional relationship between the deflection of the Fabry-Pérot cavity and intraocular pressure in the central area of the sensor, based on changes in the spacing of the sensor interference fringes caused by changes in intraocular pressure. With the advanced YOLO target detection instance segmentation and M-net deep convolutional neural network algorithm integrated in the mobile phone APP, the intraocular pressure measuring device can automatically focus, identify, and clip the sensor interference fringe area and perform real-time intraocular pressure demodulation, so that patients can measure the intraocular pressure with mobile phones by themselves at home, and signal transmission does not rely on electromagnetic energy supply, effectively avoiding signal loss caused by external factors.

The intraocular pressure sensor according to the present disclosure has stronger compatibility and universality, as it can be compatible and used in conjunction with any ophthalmic implant device in the prior art, which is helpful to further explore the feasibility of new clinical technologies integrating glaucoma monitoring, diagnosis, and treatment.

Taking the above exemplary embodiments of the present disclosure as guidance, a person skilled in the art can make various changes and modifications based on the above description, without departing from the scope and spirit of the present disclosure. The technical scope of the present disclosure is not limited to the content disclosed in the specification, but must be determined in accordance with the scope of the claims.

What is claimed is:

1. An intraocular pressure measuring method, comprising the following steps:
S1: sensing changes in intraocular pressure using a Fabry-Pérot cavity of an intraocular pressure sensor;
S2: emitting rays of light using a shooting module to the Fabry-Pérot cavity, and acquiring pictures of interference patterns using a shooting element;
S3: identifying and segmenting interference fringe areas contained in the pictures using the YOLOv11-seg model to obtain the interference fringe areas;
S4: conducting binarizing segmentation and extracting the interference fringe areas using a deep convolutional neural network, to obtain binarizing interference fringes;
S5: extracting skeletons of the binarizing interference fringes, marking orders of the fringes, and then calculating central deflections of a film of the Fabry-Pérot cavity under pressure; and
S6: acquiring intraocular pressures according to the central deflections.

2. The intraocular pressure measuring method according to claim 1, wherein the step of identifying and segmenting interference fringe areas contained in the pictures using the YOLOv11-seg model comprises:
S31: acquiring pictures of interference patterns under different environmental conditions;
S32: manually annotating the interference fringe areas in the pictures using the Labelme plug-in to obtain a txt file as a set of tags corresponding to images in a training set; and
S33: taking some images as the training set and some images as the verification set for model training, to obtain interference fringe areas.

3. The intraocular pressure measuring method according to claim 1, wherein the deep convolutional neural network comprises a left path, a right path, an encoding path, and a decoding path.

4. The intraocular pressure measuring method according to claim 3, wherein a loss function used in model training for the deep convolutional neural network comprises a focal loss function and a multi-scale structural similarity.

5. The intraocular pressure measuring method according to claim 4, wherein the focal loss function is defined as:
$${L}_{FL} ( p , y ) = {L}_{FL} ( {p}_{t} ) = - 0.5 {( 1 - {p}_{t} )}^{r} log ( {p}_{t} )$$
$$where {p}_{t} = \{ \begin{matrix}p & y = 1 \\ 1 - p & otherwise\end{matrix} ;$$
y∈{1,0} represents the true value for a sample; p∈[0,1] is the output of the model, that is, the predicted probability that the true value of the sample is 1; and r is the adjustment factor of the focal loss function.

6. The intraocular pressure measuring method according to claim 4, wherein the multi-scale structural similarity is defined as:
$$MS\_SSIM ( x , y ) = {[ {l}_{M} ( x , y ) ]}^{{α}_{M}} {\prod }_{j = 1}^{M} {{[ {c}_{j} ( x , y ) ]}^{{β}_{j}} [ {s}_{j} ( x , y ) ]}^{{γ}_{j}}$$
where M is the number of scales; α_{M}, βⱼ, γⱼ are the weights of I_{M}(x, y), cⱼ(x, y), sⱼ(x, y) respectively; and I_{M}(x, y), cⱼ(x, y), sⱼ(x, y) are the brightness similarity, contrast similarity, and structural similarity between x and y on the scales of M, i, j respectively.

7. The intraocular pressure measuring method according to claim 1, wherein a deflection distribution function of the film of the Fabry-Pérot cavity under pressure is:
$$w = {w}_{0} {( 1 - \frac{{x}^{2}}{{a}^{2}} )}^{2} {( 1 - \frac{{y}^{2}}{{a}^{2}} )}^{2} [ 1 + {c}_{1} ( \frac{{x}^{2} + {y}^{2}}{{a}^{2}} ) + {{c}_{2} ( \frac{{x}^{2} + {y}^{2}}{{a}^{2}} )}^{2} ]$$
where (x, y) are the coordinates of a point on the film, for which the origin is the center of the film; w₀ is the central deflection of the film; α is the side length of the film; and c₁, c₂ are two empirical parameters.

8. The intraocular pressure measuring method according to claim 1, wherein the step of acquiring intraocular pressures according to the central deflections comprises: using a calibrating device to calibrate the central deflections of the film of the Fabry-Perot cavity, and acquiring the intraocular pressures according to the calculated central deflections.

9. An intraocular pressure measuring device, which measures intraocular pressures using the intraocular pressure measuring method according to claim 1.

10. The intraocular pressure measuring device according to claim 9, comprising an intraocular pressure sensor (1), a shooting module (2), and a shooting element (3), wherein
the intraocular pressure sensor (1) is provided with a Fabry-Pérot cavity;
the shooting module (2) comprises a housing (21), an optical path assembly (22) arranged in the housing (21), and a light source (23) arranged outside the housing (21);
the housing (21) is provided with a shooting hole (211);
rays of light emitted by the light source (23) are transmitted to the Fabry-Pérot cavity after passing through the optical path assembly (22), and form interference patterns;
the shooting element (3) comprises a lens (31); and
the shooting hole (211) is suitable for cooperating with the lens (31) to acquire the interference patterns through the shooting element (3).
